(19) United States
(12) Patent Application Publication
(10) Pub. No.: US 2010/0042015 A1
(43) Pub. Date: Feb. 18, 2010

(54) Whole blood capillary collection system

(76) Inventor: Bradley V. Brown, Leawood, KS (US)

Correspondence Address: THOMTE LAW OFFICE, L.L.C., 2120 S. 72ND STREET, SUITE 1111, OMAHA, NE, 68124, US

(21) Appl. No.: 12/228,609

(22) Filed: Aug. 14, 2008

(51) Int. Cl. A61B 5/151

(52) U.S. Cl. 600/578

(57) ABSTRACT

A whole blood capillary collection system is described and includes an enclosure located remotely from a blood collection apparatus. A DC motor-driven vacuum pump is located within the enclosure and is operatively connected to one end of the flexible tube so as to create suction therein. The enclosure includes a switch for controlling the operation of the vacuum pump and also includes a low battery light indicator. By locating the enclosure remote from the collection apparatus, the noise level at the puncture site is reduced. The enclosure may be conveniently placed in the user's pocket or placed on a supporting surface.

### BACKGROUND OF THE INVENTION

1. Field of the Invention

This invention relates to a whole blood capillary collection system and more particularly to a whole blood capillary collection system wherein a battery-operated vacuum source is contained within an enclosure with an elongated tube being connected to the vacuum source and which extends to a blood collection apparatus located remotely therefrom.

2. Description of the Related Art

In Applicant's earlier U.S. Pat. Nos. 5,110,557 and 5,238,655 and in the collection apparatus marketed by Innovative Med Tech, the blood collection apparatus is mounted directly onto a hand-held vacuum source so that blood at the patient collection (puncture) site will be drawn into a collection tube. Although the devices of the above-identified patents and that of Innovative Med Tech have been successful, it has been found that a more conveniently useable system could be employed if the collection apparatus is positioned remotely from the vacuum source so that the vacuum source may be placed on a table, tray, etc. or placed in the pocket of the technician collecting the sample. It has been found that the instant invention makes the system more comfortable and natural for the user to operate, reduces the noise level near the patient, and eliminates the need to disinfect the device between patients.

### SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

A whole blood capillary collection system is described comprising a portable enclosure having an internal compartment with the enclosure being sized such that it may be placed in a pocket of the user. The enclosure also has a shape which enables it to be placed upon a supporting surface such as countertop, table, etc.

A 9-volt DC battery is positioned in the internal compartment of the enclosure as is a vacuum pump which is driven by a DC motor also located within the enclosure. A switch is provided at the outside surface of the enclosure and is operatively connected to the battery and the DC motor. The system also includes an elongated, flexible tube having intake and discharge ends. The discharge end of the tube is operatively connected to the vacuum pump so that suction is created in the tube. A blood collection apparatus is operatively connected to the intake end of the tube remote from the enclosure and is adapted to collect blood at a puncture site. Preferably, the enclosure also includes a low battery indicator. The system also includes a hydrophobic filter which is positioned between the discharge end of the tube and the vacuum pump.

It is therefore a principal object of the invention to provide a whole blood capillary collection system wherein a blood collection apparatus is located remotely of an enclosure and is connected thereto by means of a flexible tube.

A further object of the invention is to provide a system of the type described wherein the system is more comfortable and natural for the user to operate than prior art devices.

A further object of the invention is to provide a system of the type described wherein the vacuum pump thereof is located remotely from the blood collection apparatus to reduce the noise level near the patient.

Yet another object of the invention is to provide a system of the type described which eliminates the need to disinfect the device between patients.

These and other objects will be apparent to those skilled in the art.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is an exploded perspective view of the blood sample collection apparatus of this invention;

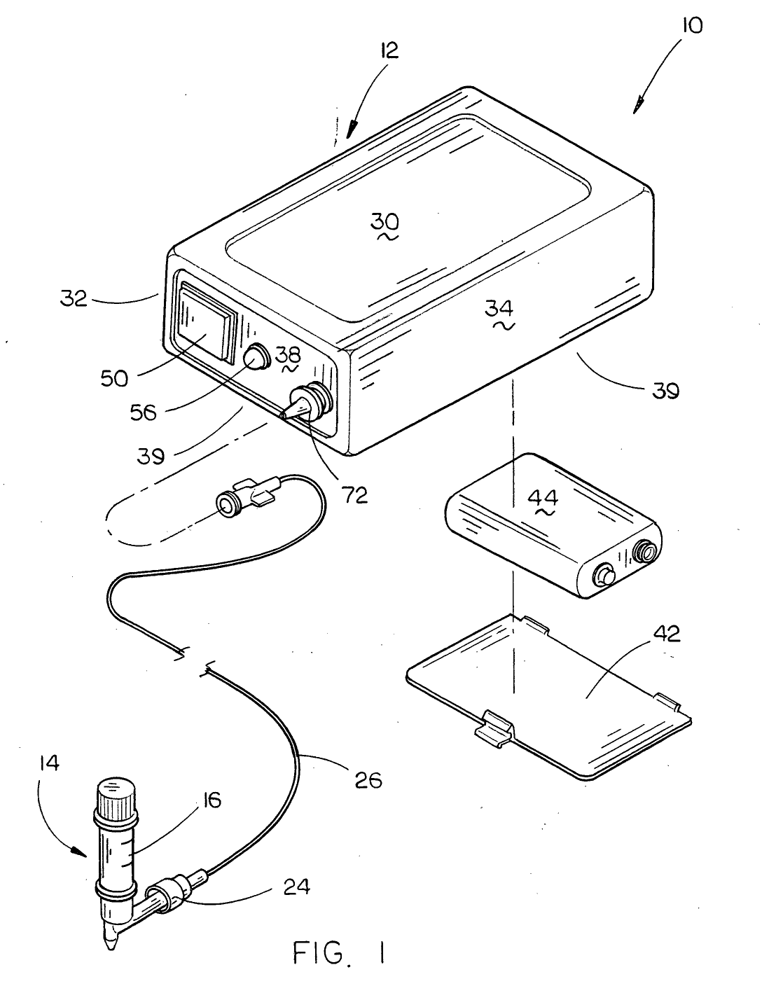

FIG. 2 is a perspective view of the system of this invention being used to collect blood from the heel of an infant; and

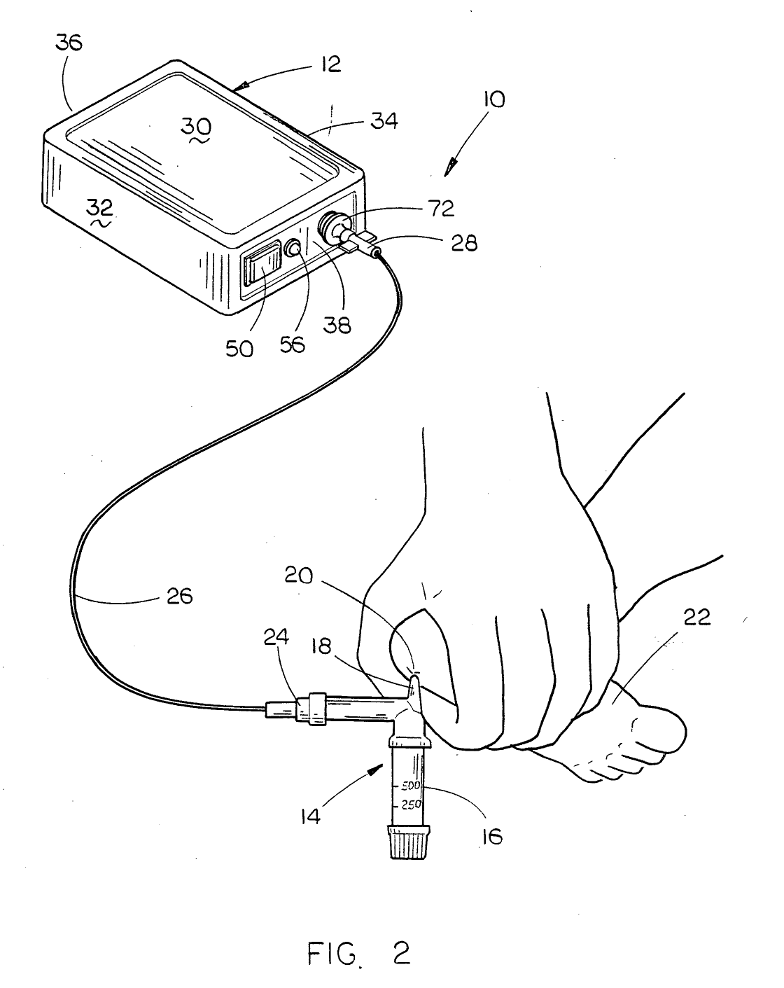

FIG. 3 is a schematic view of the circuitry of the vacuum source of this invention.

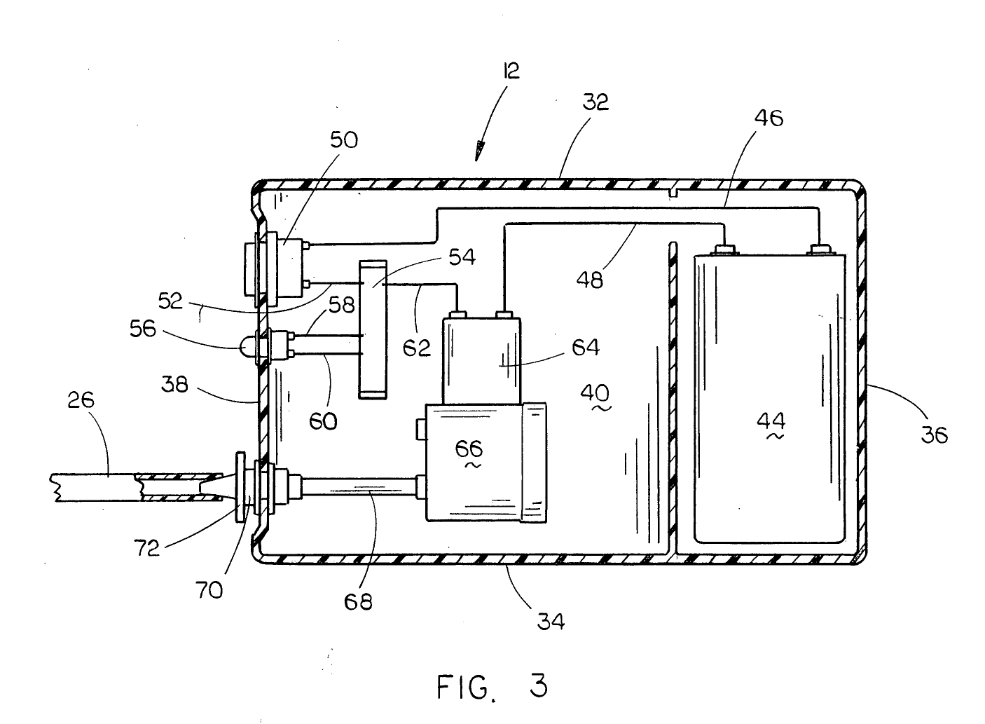

### DESCRIPTION OF THE PREFERRED EMBODIMENT

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense in that the scope of the present invention is defined only by the appended claims.

The whole blood capillary collection system of this invention is referred to generally by the reference numeral 10 and includes a vacuum source located within an enclosure 12. The numeral 14 refers to a blood collection apparatus such as disclosed in U.S. Pat. Nos. 5,110,557 and 5,238,655 or that marketed by Innovative Med Tech under the trademark Quick Draw®. Apparatus 14 includes a collection tube 16 which is in communication with a pick-up tube 18 which is adapted to collect blood at a puncture site 20 such as on the heel 22 of a patient. A male luer lock connector 24 is connected to one end of an elongated flexible tube 26. The connector 24 fits into the female luer connection of apparatus 14. The other end of tube 26 has a female luer lock connector 28 secured thereto.

Enclosure 12 includes a top wall 30, side walls 32 and 34, bottom wall 35 and end walls 36 and 38. Enclosure 12 is sized so as to be able to be placed within the user's pocket and is shaped so as to be able to be placed upon a supporting surface such as a table top, counter, tray, etc. Enclosure 12 defines an internal compartment 40, access to which is gained by means of removable cover 42 located on the bottom wall 35.

A 9-volt DC battery 44 is positioned within the compartment 40 and has leads 46 and 48 removably secured to the terminals thereof. Lead 46 is connected to a switch 50 positioned in front wall 38 of enclosure 12. Lead 52 extends from switch 50 to a circuit board 54. Circuit board 54 is attached to a low-battery indicator light positioned in front wall 38 of enclosure 12 by leads 58 and 60. Lead 62 extends from circuit board 54 to a DC motor 64 to which lead 48 is connected. Motor 64 drives a vacuum source or vacuum pump 66 having an intake tube or conduit extending therefrom. The outer end of conduit 68 has a hydrophobic filter 70 provided thereon to prevent contaminants being drawn into vacuum pump 66. A male luer connector 72 is secured to the outer end of the conduit 68 and has the connector 28 of the tube 26 secured thereto.

In operation, the enclosure 12 may be placed on a table or the like or may be placed in a pocket of the user so as to be located remote from the puncture site. By being located remotely of the puncture site, the noise level of the vacuum pump is moved from near the patient to a location remote therefrom. The system disclosed herein represents an improvement over the prior art by making it more comfortable and natural for the user to operate and eliminates the need to disinfect the device between patients. With the switch 50 activated, the pick-up tube 18 is positioned at the puncture site 20 to collect the blood therefrom. The fact that the collection apparatus 14 is connected to one end of the tube 26 rather than to a hand-held vacuum source adjacent thereto makes the collection apparatus more comfortable and natural for the user to operate.

Thus it can be seen that the invention accomplishes at least all of its stated objectives.

Although the invention has been described in language that is specific to certain structures and methodological steps, it is to be understood that the invention defined in the appended claims is not necessarily limited to the specific structures and/or steps described. Rather, the specific aspects and steps are described as forms of implementing the claimed invention. Since many embodiments of the invention can be practiced without departing from the spirit and scope of the invention, the invention resides in the claims hereinafter appended.

1. A whole blood capillary collection system, comprising:
a portable enclosure having an outside surface and an internal compartment;
a battery in said internal compartment;
a DC motor in said internal compartment;
a vacuum pump operatively driven by said DC motor;
a switch at said outside surface of said enclosure operatively electrically connected to said battery and said DC motor;
an elongated, flexible tube having intake and discharge ends;
said discharge end of said tube being operatively connected to said vacuum pump which suction is created in said tube;
said switch being operatively coupled to said DC motor for controlling the operation of said DC motor and said vacuum pump;
an a blood capillary collection apparatus operatively connected to the intake end of said tube remote from said enclosure adapted to collect blood at a puncture site.

2. The system of claim 1 wherein said enclosure is sized to permit it to be placed in a pocket of the person collecting the blood sample.

3. The system of claim 1 wherein said enclosure is configured so as to permit said enclosure to be placed on a supporting surface remote from the puncture site.

4. The system of claim 1 wherein said enclosure defines a generally rectangular block and includes a rectangular top wall member, a rectangular bottom wall member and opposite side walls and end walls joining said top and bottom wall members.

5. The system of claim 1 further including a low battery indicator on said enclosure.

6. The system of claim 1 further including a hydrophobic filter positioned between said discharge end of said tube and said vacuum pump.
